(19) United States
(12) Patent Application Publication
(10) Pub. No.: US 2024/0374619 A1
(43) Pub. Date: Nov. 14, 2024

(54) COMPOSITION FOR TREATING OPHTHALMIC CONDITIONS AND METHODS OF USE THEREOF

(71) Applicant: Charles Strickler, Newport Coast, CA (US)

(72) Inventor: Brian Stuart Murphy, Newport Coast, CA (US)

(21) Appl. No.: 18/472,584

(22) PCT Filed: Mar. 22, 2022
(86) PCT No.: PCT/US2022/021302

§ 371 Date: Sep. 22, 2023

Related U.S. Application Data
(60) Provisional application No. 63/164,735, filed on Mar. 23, 2021.

(51) Int. Cl. A61K 31/00; A61K 31/13

(52) U.S. Cl. CPC A61K 31/658; A61K 31/13

(57) ABSTRACT

A method for treating ophthalmic conditions comprising the step of administering to a subject i) a cannabinoid, and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator; and compositions thereof.

### CROSS-REFERENCE TO RELATED APPLICATIONS

This National Stage Utility Patent Application claims the benefit of and priority to International Patent Application No. PCT/2022/021302, filed on Mar. 22, 2022, the contents of which are expressly incorporated herein by this reference, and to which priority is claimed. International Patent Application No. PCT/2022/021302 claims the benefit of and priority to U.S. Provisional Patent Application No. 63/164,735, filed on Mar. 23, 2021, the contents of which are expressly incorporated herein by this reference, and to which priority is claimed.

### FIELD

One or more embodiments of the disclosure relate to the field of pharmaceutical compositions for treating an ophthalmic condition. The disclosure also relates to one or more embodiments of a method for treating an ophthalmic condition.

### BACKGROUND

Ophthalmic conditions can be painful, distracting and debilitating. Eye pain is a common symptom, and there are many potential causes, ranging from the serious, such as acute angle-closure glaucoma and optic neuritis, to the less serious, such as conjunctivitis, styes, or dry eye. Blepharitis, for example, is a chronic disorder that produces inflammation of the anterior and posterior lid margin. It can also affect the conjunctiva, tear film, and the corneal surface in advanced stages and may be associated with dry eye. Blepharitis is one of the most common ocular disorders and has no cure to date or FDA-approved treatments.

The pathophysiology of blepharitis is not well understood, but bacteria, altered meibum lipid composition and inflammation are understood as major contributors to the process. Current therapies such as warm compresses, lid cleansing, oral nutritional supplements, and oral tetracycline antibiotics present compliance difficulties and disappointing results. The inflammatory aspects of blepharitis have been treated with topical steroids and systemic tetracycline. However, the well-known side effects of steroid use and long term systemic antibiotic use make these treatment regimes less than optimal. Antibiotic ointments have been used to treat the overgrowth of normal bacterial flora in this disease, but do not address the inflammatory aspects.

There exists a need in the art for pharmaceutical compositions and methods for treating ophthalmic conditions, such as blepharitis and other conditions that result in inflammation and pain, and corresponding pharmaceutical compositions that avoid the side effects of steroids and antibiotics.

### BRIEF SUMMARY

According to one or more embodiments, disclosed herein is a method of treating an ophthalmic condition comprising administering to a subject i) a cannabinoid and ii) a modulator compound that acts as one or more of a N-methyl-D-aspartate (NMDA) receptor modulator, a 5-HT₃ receptor modulator, a nicotinic acetylcholine receptor modulator, a dopamine D2 receptor modulator, or a sigma-1 receptor modulator.

In one or more embodiments, disclosed herein is a pharmaceutical composition comprising i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator.

### Definitions

As used herein, the singular forms “a,” “an,” and “the” include plural references unless the context clearly indicates otherwise. Thus, for example, reference to “a cannabinoid” includes a single cannabinoid as well as a mixture of two or more different cannabinoid or a derivative, prodrug or analogue thereof; and reference to an “excipient” includes a single excipient as well as a mixture of two or more different excipients, and the like.

As used herein, the term “about” in connection with a measured quantity or time, refers to the normal variations in that measured quantity or time, as expected by one of ordinary skill in the art in making the measurement and exercising a level of care commensurate with the objective of measurement. In certain embodiments, the term “about” includes the recited number ±10%, such that “about 10” would include from 9 to 11, or “about 1 hour” would include from 54 minutes to 66 minutes.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

As used herein, the term “active agent” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

As used herein, the terms “therapeutically effective” and an “effective amount” refer to that amount of an active agent or the rate at which it is administered needed to produce a desired therapeutic result.

The term “subject” refers to a human or animal, who has demonstrated a clinical manifestation of an ophthalmic condition. The term “subject” may include a person or animal (e.g., a canine) who is a patient being appropriately treated by a medical caregiver for an ophthalmic condition.

The terms “treatment of” and “treating” include the administration of an active agent(s) with the intent to lessen the severity of a condition and/or a symptom.

The terms “prevention of” and “preventing” include the avoidance of the onset of a condition by a prophylactic administration of the active agent.

The term “condition” or “conditions” may refer to those conditions commonly recognized as affecting the eye or eye area, including, but are not limited to, ophthalmic conditions involving inflammation, pain, fibrosis, infection, virus, parasite, neurodegeneration, retinal ganglion cell (RGC) degeneration and/or combinations thereof, which can be treated, mitigated or prevented by a timely administration to a subject of an effective amount of a pharmaceutical composition as described herein. The condition may affect the eyelid, ocular surface, back of the eye, dermis, eyelashes, tarsal conjunctiva, mucocutaneous junction and/or meibomian glands. The condition may be at least partially caused by bacteria, virus, fungus, yeast, mites, seborrhea, rosacea, environmental exposure, hormonal dysregulation, inflammatory process, cytokine cascade and/or an autoimmune disorder. The condition may be treatable with a pharmaceutical composition as described herein. In certain embodiments, the term “condition” or “conditions” may refer to blepharitis, glaucoma, uveitis, a stye, rosacea, autoimmune disease, glandular condition, chalazion, corneal abrasion, corneal herpetic infections, corneal ulcer, dry eyes, iritis, keratitis, optic neuritis, conjunctivitis, scleritis, age-related macular degeneration, allergic conjunctivitis, eosinophilic keratitis, anterior segment scarring, blepharoconjunctivitis, a bullous disorder, cicatricial pemphigoid, conjunctival melanoma, contact lens-associated giant papillary conjunctivitis, diabetic retinopathy, episcleritis, gliosis, granuloma annulare, Graves' ophthalmopathy, intraocular melanoma, keratitis, keratoconjunctivitis pain, pinguecula, post-surgical pain, inflammation, proliferative vitreoretinopathy, pterygia, Sjögren's syndrome, Scleroderma, systemic sclerosis, vernal and/or any combination of two or more of the foregoing.

T_{1/2} refers to the time for the plasma concentration of an active agent to decrease by half.

The term “concurrently” as used herein means that a dose of one agent (e.g., a cannabinoid or a modulator) is administered prior to the end of the dosing interval of another agent (e.g., a cannabinoid or a modulator). For example, a dose of a cannabinoid with a particular dosing interval would be concurrently administered with a modulator dose when administered within the dosing interval of the cannabinoid.

The term “simultaneously” as used herein means that a dose of one agent is administered approximately at the same time as another agent, regardless of whether the agents are administered separately via the same or different routes of administration or in a single pharmaceutical composition or dosage form. For example, a dose of a cannabinoid may be administered separately from, but at the same time as, a dose of a modulator.

The term “sequentially” as used herein means that a dose of one agent is administered first and thereafter a dose of another agent is administered second. For example, a dose of a cannabinoid may be administered first, and thereafter a dose of a modulator may be administered second. The subsequent administration of the second agent may be inside or outside the dosing interval of the first agent.

### DETAILED DESCRIPTION

In one or more embodiments, described herein is a method of treating an ophthalmic condition comprising administering to a subject i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator. In certain embodiments, the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator. In particular embodiments, the modulator compound is memantine, a prodrug thereof, an analog thereof, a metabolite thereof, or a pharmaceutically acceptable salt thereof. In certain embodiments, the cannabinoid comprises one or more of a natural compound, a synthetic compound, or a semi-synthetic compound. In particular embodiments, the cannabinoid is cannabidiol (CBD) hemisuccinate and/or dronabinol.

In some embodiments, the method includes administering cannabidiol or a pharmaceutically acceptable salt thereof and memantine or a pharmaceutically acceptable salt thereof. In certain embodiments, the cannabinoid and the modulator compound are administered to the subject via the same or different routes, for example, independently, by oral (e.g., an oral solid dosage form, a tablet, a capsule, a softgel, a powder, a suspension or a solution), sublingual, parenteral, ophthalmic (e.g., liquid drops, suspension drops, topical cream, topical ointment, topical lotion or topical gel), transdermal (e.g., subcutaneous by an implantable solid, semi-solid, gel or a viscous liquid), topical (e.g., a cream, an ointment, a lotion or a gel) and/or trans-gingival routes (e.g., a tape-like strip that is applied to the gum and dissolves allowing absorption of drug directly into the bloodstream and bypassing the gastrointestinal tract and first-pass metabolism in the liver).

In certain embodiments, the ophthalmic condition affects the eyelid, ocular surface, back of the eye, dermis, eyelashes, tarsal conjunctiva, mucocutaneous junction or meibomian glands. In some embodiments, the ophthalmic condition is an inflammatory disorder, for example, one or more of blepharitis, conjunctivitis, iritis, uveitis, keratitis or corneal ulcer.

In certain embodiments, the methods treat one or more of watery eye, red eye, gritty sensation in eye, burning sensation in eye, stinging sensation in eye, itchy eyelid, red eyelid, swollen eyelid, flaking of skin around the eye, crusted eyelash, eyelid sticking, frequent blinking, sensitivity to light and blurred vision.

In certain embodiments, the methods include administering a cannabinoid, a modulator or both each independently in the form of nanoparticles, a nanoemulsion or nanocrystals.

In one or more embodiments, described herein is a pharmaceutical composition comprising i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator. In certain embodiments, the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator. In particular embodiments, the modulator compound is memantine, a prodrug thereof, an analog thereof, a metabolite thereof or a pharmaceutically acceptable salt thereof. In certain embodiments, the cannabinoid comprises one or more of a natural compound, a synthetic compound, or a semi-synthetic compound. In particular embodiments, the cannabinoid is cannabidiol hemisuccinate and/or dronabinol.

In some embodiments, the pharmaceutical composition includes cannabidiol or a pharmaceutically acceptable salt thereof and memantine or a pharmaceutically acceptable salt thereof. In certain embodiments, the cannabinoid and the modulator compound are administered to the subject via the same or different routes, for example, independently by oral (e.g., an oral solid dosage form, a tablet, a capsule, a softgel, a powder, a suspension or a solution), sublingual, parenteral, ophthalmic (e.g., liquid drops, suspension drops, topical cream, topical ointment, topical lotion or topical gel), transdermal (e.g., subcutaneous by an implantable solid, semi-solid, gel or a viscous liquid) or topical routes (e.g., a cream, an ointment, a lotion or a gel).

In certain embodiments, the ophthalmic condition affects the eyelid, ocular surface, back of the eye, dermis, eyelashes, tarsal conjunctiva, mucocutaneous junction or meibomian glands. In some embodiments, the ophthalmic condition is an inflammatory disorder, for example, one or more of blepharitis, conjunctivitis, iritis, uveitis, keratitis or corneal ulcer.

In certain embodiments, the methods treat one or more of watery eye, red eye, gritty sensation in eye, burning sensation in eye, stinging sensation in eye, itchy eyelid, red eyelid, swollen eyelid, flaking of skin around the eye, crusted eyelash, eyelid sticking, frequent blinking, sensitivity to light and blurred vision.

In certain embodiments, the at least one pharmaceutical composition is in the form of nanoparticles, a nanoemulsion or nanocrystals.

### Active Agents

According to one or more embodiments, pharmaceutical compositions as described herein include a cannabinoid, a modulator compound, or both a cannabinoid and a modulator compound. According to one or more embodiments, a suitable cannabinoid compound for pharmaceutical compositions (and methods) as described herein includes, but is not limited to, a natural cannabinoid compound (e.g., a phytocannabinoid, an endocannabinoid, a metabolite), a synthetic cannabinoid compound (e.g., chemically synthesized), a semi-synthetic cannabinoid compound (e.g., a derivative of a natural cannabinoid) or a combination of any two or more of the foregoing. In some embodiments, the cannabinoid compound includes one or more of an isolate, a full-spectrum cannabinoid or a broad spectrum cannabinoid. A full-spectrum cannabinoid compound includes all biologically active constituents present in a cannabis plant, that is, the cannabinoid compound may include plant material and/or all compounds (e.g., all cannabinoids) of a cannabis plant. In one or more embodiments, the cannabinoid compound includes plant material from a Cannabis Sativa L. plant, a Cannabis Indica plant, a Cannabis Ruderalis plant, a Cannabis hybrid of any two of the foregoing, a derivative thereof, a prodrug thereof, an analog thereof, a metabolite thereof and/or a salt thereof.

In one or more embodiments, the cannabinoid may be an anti-inflammatory. Without being bound by any particular theory, it is believed that cannabinoids may be useful to help manage acute or chronic pain and inflammation (i.e., as an anti-inflammatory agent) associated with, for example, ophthalmic conditions. Agonist-activated cannabinoid receptors can modulate nociceptive thresholds, inhibit the release of pro-inflammatory molecules and display synergistic effects with other systems that influence analgesia, particularly the endogenous opioid system. As such, cannabinoids can act on inflammation through mechanisms different from those of agents such as nonsteroidal anti-inflammatory drugs (NSAIDs). As a class, the cannabinoids also may be free from the adverse effects associated with NSAIDs. Their clinical development may provide a new approach to treatment of diseases characterized by acute and chronic inflammation and subsequent fibrosis.

In some embodiments, the cannabinoid may be an anti-fibrotic agent. Fibrous connective tissue develops as a reparative response to injury or damage. Fibrosis may refer to connective tissue deposition that occurs as part of normal healing or to excess tissue deposition that occurs as a pathological process. An example of fibrosis is pre-retinal fibrosis. For example, pre-retinal fibrosis is an ocular condition that affects the retina, that is, a thin membrane of scar-like tissue covers the surface of the macula. Without being bound by any particular theory, it is believed that certain cannabinoids, for example, cannabidiol (i.e., natural, synthetic or semi-synthetic), may reduce fibrosis in tissue and scarring (e.g., externally and/or internally). Some cannabinoids, such as tetrahydrocannabinol (THC), may increase fibrosis of tissue and scarring.

In one or more embodiments, the cannabinoid may be an anti-infective agent. Cannabinoids, such as Δ⁹-THC and CBD, have been found to have antimicrobial, bacteriostatic and bactericidal activity, for example, against Gram-positive pathogens (e.g., methicillin-resistant Staphylococcus aureus or MRSA isolates). In addition to THC and CBD, the cannabinoids cannabidiolic acid (CBDA), cannabigerol (CBG), cannabigerolic acid (CBGA) and cannabinol (CBN) have been found to have antibacterial activity against multidrug-resistant (MDR) S. aureus isolates.

In some embodiments, the cannabinoid may be a neuroprotective agent. The biological effects of cannabinoids are mediated by two cannabinoid receptors: CB1 and CB2. The CB1 receptor is the prominent type in the central nervous system (CNS) and may be therapeutic for neuropsychological disorders and neurodegenerative diseases. For example, overactivation of glutamate N-methyl-D-aspartate receptor (NMDAR) increases the cytosolic concentrations of calcium and zinc, which significantly contributes to neural death. Because cannabinoids prevent the NMDAR-mediated increase in cytosolic calcium, they also may control the rise of toxic free zinc ions, as well as the processes implicated in this phenomenon. It has been found that the CB1 receptor can regulate NMDAR function when the receptor is coupled to the histidine triad nucleotide-binding protein 1 (HINT1). In another example, THC may preserve retinal ganglian cells. The retina is anatomically and developmentally an extension of the CNS, and the retina and the brain are connected by the optic nerve, the axons of the ganglion cells, through the lateral geniculate nucleus. Cannabinoids have shown neuroprotective effects in different models of retinal neurodegeneration.

Suitable cannabinoid compounds for pharmaceutical compositions as described herein include, but are not limited to, cannabinol, cannabidiol, cannabidiol hemisuccinate, cannabidiol valine hemisuccinate, CBD-Di-Alaninate-Di-Hemisuccinate, CBD-Di-Valinate-Di-HS, CBD-Mono-Valinate-Mono-Hemisuccinate, CBD-monovalinate-dihemisuccinate, cannabigerol, cannabichromene, cannabidiolic acid, cannabigerolic acid, Δ⁹-tetrahydrocannabinol, Δ⁸-tetrahydrocannabinol, 11-hydroxy-tetrahydrocannabinol, 11-hydroxy-Δ⁹-tetrahydrocannabinol, Δ¹¹-tetrahydrocannabinol, tetrahydrocannabivarin, anandamide, 2-arachidonoylglyerol, dronabinol, levonantradol, nabilone, a derivative thereof, a prodrug thereof, an analog thereof, a metabolite thereof and/or a pharmaceutically acceptable salt thereof. In some embodiments, the pharmaceutical composition is free of tetrahydrocannabinol, a derivative thereof, a prodrug thereof, an analog thereof, a metabolite thereof and/or a pharmaceutically acceptable salt thereof. In one or more embodiments, the cannabinoid compound includes one or more of THC, CBD, CBDA, CBG, CBGA, CBN, a derivative thereof, a prodrug thereof, an analog thereof, a metabolite thereof and/or a pharmaceutically acceptable salt thereof.

Suitable natural cannabinoid compounds for pharmaceutical compositions (and methods) as described herein include, but are not limited to, the phytocannabinoids Δ⁹-tetrahydrocannabinol (Δ⁹-THC), Δ⁸-tetrahydrocannabinol (Δ⁸-THC), cannabinol (CBN), cannabidiol, cannabigerol, cannabichromene (CBC), cannabivarin (CBV), cannabidivarin (CBDV), cannabinodiol (CBND), cannabielsion (CBE), cannabicyclol (CBL), cannabitriol (CBT) cannabinol, cannabigerol (CBG), cannabidiolic acid, cannabigerolic acid, the endocannabinoids anandamide (AEA) or 2-arachidonoylglyerol (2-AG), the metabolites 11-hydroxy-tetrahydrocannabinol, 11-hydroxy-Δ⁹-tetrahydrocannabinol, Δ¹¹-tetrahydrocannabinol, tetrahydrocannabivarin, N-desmethylclobazam, hydroxylated 7-COOH derivatives of CBD, and/or any combination of two or more of the foregoing. In one or more embodiments, a natural cannabinoid compound suitable for pharmaceutical compositions described herein includes one or more of THC, CBD, CBDA, CBG, CBGA, CBN and/or a metabolite thereof.

Suitable synthetic cannabinoid compounds for pharmaceutical compositions (or methods) according to at least one embodiment herein include, but are not limited to, dronabinol (e.g., Marinol®, Syndros®), nobiline (e.g., Ceasamet®), synthetic cannabidiol (e.g., Epidiolex®) a derivative thereof, a prodrug thereof, an analog thereof, a metabolite thereof, a pharmaceutically acceptable salt thereof and/or any combination thereof. In one or more embodiments, the cannabinoid compound is any cannabinoid made in a laboratory (e.g., through chemical synthesis).

Suitable semi-synthetic cannabinoid compounds for pharmaceutical compositions (or methods) described herein include, but are not limited to, a derivative of a natural cannabinoid) Δ⁹-tetrahydrocannabivarin (THCV), an analogue of a natural cannabinoid, a homologue of a natural cannabinoid, a hydrogenated cannabinoid, cannabidiol-dimethylheptyl, a metabolite of a natural or synthetic cannabinoid, a derivative thereof, a prodrug thereof, an analog thereof, a metabolite thereof, a pharmaceutically acceptable salt thereof and/or any combination thereof.

The modulator compound of the pharmaceutical composition (and methods) as described herein may include one or more of an NMDA receptor modulator, a 5-HT₃ receptor modulator, a nicotinic acetylcholine receptor modulator, a dopamine D2 receptor modulator, a sigma-1 receptor modulator and/or a combination thereof. In at least one embodiment, the NMDA receptor modulator is an agonist, an antagonist, or a mixed agonist-antagonist at one or more of the NMDA receptor modulator, the 5-hydroxytryptamine (5-HT₃) receptor modulator, the nicotinic acetylcholine receptor modulator, the dopamine D2 receptor modulator, and/or the sigma-1 receptor modulator.

NMDA receptor modulators (e.g., glutamate modulators) mediate a slow component of excitatory synaptic transmission, and NMDA receptor dysfunction has been implicated in numerous neurological disorders. For example, NMDA receptors located in the retinal ganglion cells play a role in visual processing including contrast coding. The NMDA receptor modulates synaptic plasticity in the visual cortex, a process that contributes to visual function. According to at least one embodiment, the modulator compound is a NMDA receptor antagonist. Non-limiting examples of NMDA receptor antagonists include a glutamatergic NMDA receptor antagonist, memantine and/or combinations thereof.

In some embodiments, the modulator compound is memantine, a prodrug thereof, an analog thereof, a metabolite thereof or a pharmaceutically acceptable salt thereof. Memantine hydrochloride is approved for treating moderate to severe dementia in adults with Alzheimer's disease. It is believed that memantine treatment, when started in the early phase of the glaucomatous process, may help preserve the retinal ultrastructure and thus prevent neuronal injury in glaucoma. Glutamate-induced excitotoxicity is implicated in glaucoma and NMDA receptor antagonism, such as memantine, has been advocated as a potential strategy for retinal ganglion cell (RGC) preservation. Without being bound by any particular theory, it is believed that co-administration of a pharmaceutical composition containing both a cannabinoid (e.g., CBD) and memantine may be useful to treat ophthalmic conditions. For example, the co-administration of a cannabinoid and memantine may act synergistically to produce a therapeutic effect greater than the additive effect of each component alone. In embodiments, the co-administration of a pharmaceutical composition containing a cannabinoid (e.g., CBD) and memantine may act synergistically to reduce inflammation associated with ophthalmic conditions.

In at least one embodiment, the modulator is a 5-HT receptor agonist, antagonist or mixed agonist-antagonist. 5-HT receptor modulators may be useful in the treatment of ocular conditions such as glaucoma. For example, a topical application of ketanserin, a 5-HT₂ receptor antagonist with additional alpha-1 adrenoceptor blocking activity, may lower intraocular pressure. In another example, 5-HT₃ receptors are present in the retina. A selective 5-HT₃ agonist or a selective antagonist may provide neuroprotection to the retina. According to at least one embodiment, the modulator is a 5-HT₃ receptor antagonist. Non-limiting examples of 5-HT₃ receptor antagonists as a modulator for pharmaceutical compositions according to embodiments herein include dolasetron, granisetron, ondansetron, palonosetron or any combination of two or more of the foregoing.

In at least one embodiment, the modulator is a nicotinic acetylcholine receptor agonist, antagonist or mixed agonist-antagonist. Acetylcholine (ACh) activates both nicotinic and muscarinic acetylcholine receptors (AChRs). One class includes heteromeric nicotinic AChR subtypes comprised of the α2-α6 and β2-β4 units with high agonist affinity but insensitivity to snake toxin α-bungarotoxin (αBgt); a second class includes homomeric (i.e., α7, α8, or α9) or heteromeric pentamers (i.e., combined α7, α8, α9, or α10 subunits) with lower agonist affinity but with high sensitivity to αBgt. Without being bound by any particular theory, it is believed that the α7 agonist may have a neuroprotective effect in the retina. Besides mediating visual processing, retinal nAChRs may influence refractive development and ocular pathologies such as neovascularization. It is further believed that nicotinic acetylcholine receptor agonists prevent loss of retinal ganglion cells in a glaucoma model. Further, nicotinic receptors are involved in eye growth control. Nicotinic antagonists may affect the development of form-deprivation myopia and perhaps the growth of nongoggled eyes. In some embodiments, the modulator may include a nicotinic acetylcholine receptor antagonist. Suitable nicotinic acetylcholine receptor antagonists include, but are not limited to, chlorisondamine, mecamylamine and/or combinations thereof.

In some embodiments, the modulator is a dopamine D2 receptor agonist, antagonist or mixed agonist-antagonist. Dopamine receptors play important roles in the activity dependent synaptic plasticity in CNS and multiple subtypes of dopamine receptors are expressed by retinal neurons. D1 dopamine receptors have been shown to regulate the developmental enhancement of ERG b-wave and the light response gain between bipolar and ganglion/amacrine cells. It has been found that deletion of the dopamine D2 receptor has opposite effect on the inner retinal light response in comparison with D1 dopamine receptor mutation, it preferentially regulates the retinal light responses after eye opening, and effects induced by mutation of the D2 dopamine receptor on ERG are light-sensitive. In at least one embodiment, the modulator compound may be a dopamine D2 receptor agonist. Suitable dopamine D2 receptor agonists include, but are not limited to, dopamine, ibopamine (dopamine analog), fenoldopam, bromocriptine (dopaminergic agonist with higher affinity for D2 than for D1-receptors) and/or combinations thereof.

In some embodiments, the modulator is a sigma-1 receptor agonist, antagonist or mixed agonist-antagonist. Retinal degenerative diseases are a major cause of untreatable blindness. A target for treatment of retinal disease is the transmembrane protein Sigma 1 Receptor (Sig1R). Without being bound by any particular theory, it is believed that Sig1R plays a key role in modulating retinal cellular stress and that it holds great promise as a target in retinal neurodegenerative disease. According to at least one embodiment, the modulator of pharmaceutical compositions (and methods) described herein includes a sigma-1 receptor agonist. Suitable sigma-1 receptor agonists include, but are not limited to, (+)-pentazocine, (+)-SKF10,047, 2-morpholin-4-ylethyl 1-phenylcyclohexane-1-carboxylate and 1-[2-(3,4-dimethoxyphenyl)ethyl]-4-(3-phenylpropyl)piperazine) and/or a combination of any two or more of the foregoing.

In one or more embodiments, the pharmaceutical composition includes cannabidiol, cannabidiol hemisuccinate, cannabidiol valine hemisuccinate, CBD-Di-Alaninate-Di-Hemisuccinate, CBD-Di-Valinate-Di-HS, CBD-Mono-Valinate-Mono-Hemisuccinate, CBD-monovalinate-dihemisuccinate, a derivative thereof, a prodrug thereof, an analog thereof, a metabolite thereof, a pharmaceutically acceptable salt thereof and/or any combination thereof; and memantine, a derivative thereof, a prodrug thereof, an analog thereof, a metabolite thereof, a pharmaceutically acceptable salt thereof and/or any combination thereof.

In some embodiments, the pharmaceutical composition per unit dose comprises a cannabinoid as described in one or more embodiments herein in an amount of about 2 mg to about 50 mg, about 2 mg to about 40 mg, about 5 mg to about 30 mg, about 2 mg to about 25 mg, about 10 mg to about 20 mg, about 5 mg to about 20 mg, about 10 mg to about 20 mg, about 15 mg to about 25 mg, or about 15 mg to about 20 mg or any individual amount or sub-range within these ranges. According to one or more embodiments, the pharmaceutical composition comprises the cannabinoid in an amount of about 0.1 percent by weight (wt %) to about 20 wt %, about 0.5 wt % to about 15 wt %, about 1 wt % to about 10 wt %, about 2 wt % to about 5 wt %, about 0.1 wt % to about 10 wt %, about 0.5 wt % to about 10 wt %, or any individual wt % or sub-range within these ranges based on the total weight of the composition.

In one or more embodiments, the pharmaceutical composition per unit dose comprises a modulator as described in one or more embodiments herein in an amount of about 2 mg to about 50 mg, about 2 mg to about 40 mg, about 5 mg to about 30 mg, about 2 mg to about 25 mg, about 10 mg to about 20 mg, about 5 mg to about 20 mg, about 10 mg to about 20 mg, about 15 mg to about 25 mg, or about 15 mg to about 20 mg or any individual amount or sub-range within these ranges. According to one or more embodiments, the pharmaceutical composition comprises the modulator in an amount of about 0.1 wt % to about 20 wt %, about 0.5 wt % to about 15 wt %, about 1 wt % to about 10 wt %, about 2 wt % to about 5 wt %, about 0.1 wt % to about 10 wt %, about 0.5 wt % to about 10 wt %, or any individual wt % or sub-range within these ranges based on the total weight of the composition.

In at least one embodiment, the pharmaceutical composition per unit dose includes a cannabinoid (e.g., CBD) and a modulator (e.g., memantine) as described in one or more embodiments herein each individually present in an amount of about 2 mg to about 50 mg, about 2 mg to about 40 mg, about 5 mg to about 30 mg, about 2 mg to about 25 mg, about 10 mg to about 20 mg, about 5 mg to about 20 mg, about 10 mg to about 20 mg, about 15 mg to about 25 mg, or about 15 mg to about 20 mg or any individual amount or sub-range within these ranges. According to one or more embodiments, the pharmaceutical composition includes a weight ratio of the cannabinoid (e.g., CBD) to the modulator (e.g., memantine) of about 1:100 to about 100:1, about 1:50 to about 50:1, about 1:25 to about 25:1, about 1:20 to about 20:1, about 1:15 to about 15:1, about 1:10 to about 10:1, about 1:5 to about 5:1, about 1:2 to about 2:1 or any individual weight ratio or sub-range within these ranges.

In some embodiments, the pharmaceutical composition according to embodiments herein includes a cannabinoid (e.g., CBD) and a modulator (e.g., memantine) each individually present in an amount of about 0.1 wt % to about 20 wt %, about 0.5 wt % to about 15 wt %, about 1 wt % to about 10 wt %, about 2 wt % to about 5 wt %, about 0.1 wt % to about 10 wt %, about 0.5 wt % to about 10 wt %, or any individual wt % or sub-range within these ranges based on the total weight of the composition. According to one or more embodiments, the pharmaceutical composition includes a weight ratio of the cannabinoid (e.g., CBD) to the modulator (e.g., memantine) of about 1:100 to about 100:1, about 1:50 to about 50:1, about 1:25 to about 25:1, about 1:20 to about 20:1, about 1:15 to about 15:1, about 1:10 to about 10:1, about 1:5 to about 5:1, about 1:2 to about 2:1 or any individual weight ratio or sub-range within these ranges.

In one or more embodiments, pharmaceutical compositions described herein can include a supplement, a derivative thereof, a prodrug thereof, an analog thereof, a metabolite thereof and/or a salt thereof. Suitable supplements include, but are not limited to, an amino acid, a vitamin, a mineral, an herb and/or any combination thereof. Suitable amino acids include, but are not limited to, n-acetyl cysteine (NAC), cysteine, glutathione, glycine, L-alanine, β-alanine, α-aminoadipic acid, α-aminobutyric acid, γ-aminobutyric acid, α-aminoisobutyric acid, arginine, asparagine, aspartic acid, citrulline, creatine, glutamic acid, histidine, cystine, leucine, lysine, norleucine, ornithine, phenylalanine, phosphoserine, sarcosine, threonine, valine, L-theanine and/or any combination of two or more of the foregoing. According to some embodiments, pharmaceutical formulations include a cannabinoid (e.g., CBD), a modulator (e.g., memantine) and an amino acid (e.g., NAC). NAC is an acetylated derivative of the natural amino acid, L-cysteine and possesses mucolytic, anti-collagenolytic and antioxidant properties. NAC modulates the cellular redox status to influence inflammatory pathways, leading to decreased nuclear factor-kappa B activity, which regulates several proinflammatory genes that regulate the inflammation pathways. One or more pharmaceutical compositions according to embodiments herein can include a cannabinoid (e.g., CBD), a modulator (e.g., memantine) and NAC. Without being bound by any particular theory, the NAC may provide an additive or synergistic effect in treating ophthalmic conditions including inflammation.

The amino acid may be present in the pharmaceutical formulation per unit dose in an amount of about 1 wt % to about 10 wt %, about 2 wt % to about 7 wt %, about 4 wt % to about 5 wt %, or any individual wt % or sub-range within these ranges.

Suitable vitamins for pharmaceutical formulations according to embodiments herein include, but are not limited to, one or more of a retinol ester, a retinal ester or a retinyl ester (collectively vitamin A), beta-carotene, thiamine (vitamin B1), L-ascorbic acid (vitamin C), one or more of α-tocopherol, β-tocopherol, γ-tocopherol, Δ-tocopherol, α-tocotrienol, β-tocotrienol, γ-tocotrienol or Δ-tocotrienol (collectively vitamin E), copper (e.g., cupric oxide), zinc (e.g., zinc oxide), lutein, zeaxanthin, omega-3 fatty acids and/or combinations of any two or more of the foregoing. Each vitamin individually may be present in the pharmaceutical formulation per unit dose in an amount of about 1 mg to about 1,000 mg, about 1 IU to about 1,000 IU, or any individual amount or sub-range within these ranges.

Suitable minerals for pharmaceutical compositions according to embodiments herein include, but are not limited to, calcium, phosphorus, potassium, sodium, chloride, magnesium, iron, zinc, iodine, sulfur, cobalt, copper, silver, fluoride, manganese, selenium and/or a combination of any two or more of the foregoing.

Suitable herbs for pharmaceutical compositions according to embodiments herein include, but are not limited to, phytonutrients, baicalin, cerium trichloride, cerium oxide, coenzyme Q10, curcumin, epigallocatechin gallate (EGCG), green tea extract, resveratrol, ursolic acid, a fruit extract, a vegetable extract, an herb extract, curry murraya koenigil leaf, Psidium guajava, sesania grandiflora, emblic, sea kelp, mushroom, lemon peel, holy basil leaf, annatto, moringa leaf, apple, beet root, broccoli, carrot, spinach, tomato, strawberry, cherry, blackberry, green bell pepper, brussels sprout, ginger, blueberry, garlic, green onion, raspberry, parsley, cauliflower, red cabbage, asparagus, celery, cucumber, kale, peppermint leaf, or any combination of two or more of the foregoing. According to embodiments, the herbs may be present in the pharmaceutical formulation in an amount of about 10 mg to about 2000 mg, or any individual amount or sub-range within this range.

### Pharmaceutically Acceptable Excipients

Pharmaceutical compositions according to one or more embodiments herein can further include one or more pharmaceutically acceptable excipients. Examples of possible pharmaceutically acceptable excipients are described in the Handbook of Pharmaceutical Excipients, American Pharmaceutical Association (2012), which is incorporated by reference herein. Suitable excipients for pharmaceutical compositions according to various embodiments herein include, but are not limited to, plasticizers, colorants, lubricants, thermal lubricants, antioxidants, buffering agents, disintegrants or granulating agents, binding agents, diluents, glidants, anti-adherants, sweeteners, chelating agents, granulating agents, bulking agents, flavorants, surfactants, solubilizers, stabilizers, hydrophilic polymers, hydrophobic polymers, waxes, lipophilic materials, absorption enhancers, preservative, absorbent, cross-linking agents, bioadhesive polymers, pore formers, osmotic agents, polycarboxylic acids, and combinations of any two or more of the foregoing.

Examples of suitable binding agents include, but are not limited to, cellulosic polymers (e.g., hydroxypropylmethylcellulose, hydroxypropylcellulose, hydroxyethylcellulose, etc.), polyethylene glycol, an acrylic polymer, an acrylic copolymer, a graft copolymer of polyvinyl alcohol and polyethylene glycol, a polyvinyl alcohol, alginic acid, sodium alginate, starch, pregelatinized starch, sucrose, guar gum, salts thereof, derivatives thereof and combinations thereof. Additional binders include, but are not limited to, natural or synthetic waxes, fatty alcohols (e.g., lauryl, myristyl, stearyl, cetyl or cetostearyl alcohol), fatty acids, including, but not limited to, fatty acid esters, fatty acid glycerides (e.g., mono-, di-, and triglycerides), hydrogenated fats, hydrocarbons, stearic acid, hydrophobic and hydrophilic materials having hydrocarbon backbones, acacia, tragacanth, sucrose, gelatin, glucose, cellulose materials (e.g., methylcellulose and sodium carboxymethylcellulose (e.g., Tylose™)) magnesium aluminum silicate, polysaccharide acids, bentonites, polyvinylpyrrolidone (povidone), polymethacrylates, and pregelatinized starch (such as National™ 1511 and Starch 1500). Suitable waxes include, for example, beeswax, glycowax, castor wax, carnauba wax and other wax-like substances. A “wax-like” substance is defined as any material which is normally solid at room temperature and has a melting point of from about 30° C. to about 100° C.

Additional examples of binders which may be used include, but are not limited to, digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils, natural and synthetic waxes and polyalkylene glycols. In certain embodiments, hydrocarbons having a melting point of between 25° C. and 90° C. may be included. Of the long-chain hydrocarbon binder materials, fatty (aliphatic) alcohols can be incorporated into the mixture according to certain embodiments.

Examples of suitable disintegrants include, but are not limited to, sodium starch glycolate, clays (such as Veegum™ HV), celluloses (such as purified cellulose, methylcellulose, sodium carboxymethylcellulose, and carboxymethylcellulose), cross-linked sodium carboxymethylcellulose, starch, cross-linked polyvinylpyrrolidone (e.g., crospovidone), alginates, cornstarches and pre-gelatinized corn starches (such as National™ 1551 and National™ 1550), gums (such as agar, guar, locust bean, pectin, and tragacanth) and mixtures thereof. Disintegrants can be added at any suitable step during the preparation of the pharmaceutical compositions, such as prior to granulation or during a lubrication step prior to compression or encapsulation.

Suitable bulking agents include, but are not limited to, starches (e.g., corn starch), microcrystalline cellulose, lactose (e.g., lactose monohydrate), sucrose, dextrose, mannitol, calcium phosphate and dicalcium phosphate.

According to certain embodiments, the pharmaceutical compositions may include a plasticizer. Plasticizers may interact with hydrophobic materials resulting in a lower viscosity of the mixture as compared to the mixture without the plasticizer when measured under the same conditions. Certain plasticizers may lower the glass transition temperature (Tg) of hydrophobic materials. Suitable plasticizers include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers may include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate.

In at least one embodiment, the pharmaceutical composition includes a glidant. A glidant is an excipient that improves the flow characteristics of a compressible powder such as tablet ingredients or granules. Suitable glidants include, but are not limited to, silicon dioxide, colloidal silicon dioxide and the like.

Suitable diluents useful in pharmaceutical compositions as described herein include, but are not limited to, lactose (e.g., lactose (anhydrous), lactose (spray dried), lactose monohydrate), starch (e.g., directly compressible starch), mannitol, sorbitol, dextrose monohydrate, microcrystalline cellulose, dibasic calcium phosphate dihydrate, sucrose-based diluents, confectioner's sugar, monobasic calcium sulfate monohydrate, calcium sulfate dihydrate, calcium lactate trihydrate granular, dextrates (e.g., Emdex™), dextrose (e.g., Cerelose™) inositol, hydrolyzed cereal solids such as the Maltrons™ and Mor-Rex™, amylose, powdered cellulose (e.g., Elcema™), calcium carbonate, glycine, bentonite, polyvinylpyrrolidone, and the like.

Suitable lubricants include, but are not limited to, glyceryl behenate (Compritol™ 888), metallic stearates (e.g., magnesium, calcium and sodium stearates), stearic acid, hydrogenated vegetable oils (e.g., Sterotex™), talc, waxes such as beeswax and carnauba wax, silica, fumed silica, colloidal silica, calcium stearate, long chain fatty alcohols, boric acid, sodium benzoate and sodium acetate, sodium chloride, DL-Leucine, polyethylene glycols (e.g., Carbowax™ 4000 and Carbowax™ 6000), sodium oleate, sodium benzoate, sodium acetate, sodium lauryl sulfate, sodium stearyl fumarate (Pruv™), magnesium lauryl sulfate, stearic acid, stearyl alcohol, mineral oil, paraffin, micro crystalline cellulose, glycerin, propylene glycol and combinations thereof.

Suitable anti-adherents include, but are not limited to, talc, cornstarch, colloidal silicone dioxide (Cab-O-Sil™), DL-Leucine, sodium lauryl sulfate, and metallic stearates.

Other excipients (such as colorants, flavorants and sweeteners) can be utilized in embodiments of the pharmaceutical compositions where they impart little to no deleterious effect on the stability of the pharmaceutical composition.

In certain embodiments, the pharmaceutical composition may include a film coat. The film coat may include, but is not limited to, hydroxypropylmethylcellulose, polyethylene glycol, polyvinyl alcohol or a mixture of any two or more thereof.

### Dosage Forms

According to some embodiments, pharmaceutical compositions as described herein are formulated for ophthalmic administration. Suitable dosage forms for ophthalmic administration include, but are not limited to, eye drops, ointment, in situ gel, insert, contact lens, bioerodable film, biodegradable film, implant, punctal plug, mucoadhesive polymer, fornix ring, injectable fluid, a solution, a suspension, nanoparticles, nanocrystals, nanoemulsions and/or any combination of two or more of the foregoing. Example eye drops, ointments, in situ gels, injectable fluids, solutions and suspensions can include a cannabinoid (e.g., CBD) and/or a modulator (e.g., memantine) and one or more excipient chosen from a pH adjusting component (e.g., hydrochloric acid, sodium hydroxide), a cosolvent, an emulsifier, a polymer, a co-polymer, sodium alginate, a penetration enhancer, a preservative, an emollient, a tonicity adjusting component, polyethylene glycol, polyvinyl alcohol, propylene glycol, povidone, water, saline, normal saline, glycerin, mineral oil, sodium borate, boric acid, sodium chloride, calcium chloride, magnesium chloride, potassium chloride, potassium sorbate, sodium bicaronatemannitol, sodium phosphate, carboxymethylcellulose, hyaluronan, hyaluronic acid, dextrose, dextran, glycerin, hypromellose, hydroxyethylcellulose, hydroxypropylmethylcellulose, a polysaccharide, β-d-mannuronic acid and α-1 glucuronic acid residues, gellan gum, glycerin, mineral oil, castor oil, ethylenediaminetetraacetic acid (EDTA), poloxamer, white petrolatum, benzalkonium chloride, edetate disodium, anhydrous liquid lanolin, erythritol, levocarnitine, flaxseed oil, and/or combinations of any two or more of the foregoing. The cannabinoid and/or modulator in the eye drop formulation may be in the form of nanoparticles, nanocrystals or a nanoemulsion.

Examples of ocular inserts, implants, punctal plugs and fornix rings according to embodiments herein may include sterile, thin, multilayered, drug-impregnated, drug-coated, solid and/or semisolid devices placed into the cul-de-sac or conjuctival sac, whose size and shape are especially designed for ophthalmic application. They may be composed of a polymeric support containing a pharmaceutical composition and/or a film of a pharmaceutical composition according to embodiments herein. The ocular inserts may be formulated for immediate release, controlled release or modified release (e.g., containing both an immediate release component and a controlled release component). In one or more embodiments, the drug can later be incorporated as dispersion or a solution in the polymeric support. Ocular inserts may be biodegradable or non-biodegradable, bioresorbable or non-bioresorbable, bioerodible or non-bioerodible.

Example contact lenses according to embodiments herein include soft contact lenses, or hard contact lenses impregnated and/or coated with a pharmaceutical composition according to embodiments herein. The contact lenses may be formulated for immediate release, controlled release or modified release (e.g., containing both an immediate release component and a controlled release component). In one or more embodiments, the drug can later be incorporated as dispersion or a solution in the lenses. Contact lenses may be biodegradable or non-biodegradable, bioresorbable or non-bioresorbable, bioerodible or non-bioerodible.

Mucoadhesive polymers for use in the treatment of ophthalmic conditions may be formulated to contain a pharmaceutical composition according to embodiments herein. Mucoadhesive polymers can improve drug retention by mucoadhesion of a polymer excipient that attaches to, for example, the corneal rather than conjunctival mucin. The polymer-mucin bond may be used to entrap soluble, colloidal and/or particulate material containing the pharmaceutical composition on the eye surface. As such, these polymers can increase the precorneal residence of a cannabinoid and/or a modulator at the ocular site. Viscosity-enhancing agents and mucoadhesive polymers may be used to increase the bioavailability, precorneal residence time and controlled release with reduced dosing frequency without causing any visual disturbances in the treatment of severe ocular diseases. Suitable mucoadhesive polymers include, but are not limited to, a natural polymer, a synthetic polymer, chitosan, alginate, gellan gum, guar gum, carbomer, Eudragit and/or any combination of two or more of the foregoing. Alginate is a suitable anionic polymer that acts as a penetration enhancer and has a high mucoadhesive strength. A pharmaceutical composition according to embodiments herein may be impregnated in, coated on and/or a applied on the mucoadhesive polymer.

The cannabinoid (e.g., CBD), modulator component (e.g., memantine), excipients and/or supplements according to embodiments herein may be in the form of nanoparticles, nanocrystals, nanoemulsions (e.g., a suspension of nanoparticles or nanocrystals in a fluid), nanocapsules, nanospheres and/or combinations thereof. Polymeric NPs have shown great potential for targeted delivery of drugs for the treatment of several diseases. Nanoparticles, nanocrystals, nanocapsules and nanospheres for use in pharmaceutical compositions according to embodiments herein may have a particle size distribution of less than about 1,000 nm, less than about 500 nm, less than about 250 nm, less than about 200 nm or any individual size or sub-range within these ranges as measured by dynamic light scattering and 1H-NMR. In some embodiments, the nanoparticles or nanocrystals have a mean size of about 1 nm to about 1,000 nm or any individual size or sub-range within this range. In some embodiments, pharmaceutical compositions according to embodiments herein may include memantine loaded polylactide-co-glycolide (PLGA)—polyethylene glycol (PEG) nanoparticles, which may be efficacious in the treatment of glaucoma. The drug loading and/or incorporation of the memantine loaded PLGA-PEG nanoparticles may be about 1 mg/mL to about 20 mg/mL, about 4 mg/mL to about 10 mg/mL, or any individual concentration or sub-range within these ranges.

In some embodiments, the pharmaceutical compositions disclosed herein are in solid oral dosage form such as a pharmaceutically acceptable tablet or capsule. According to embodiments, mixtures or blends of dried particles, nanoparticles, nanocrystals and/or granules contain, for example, a cannabinoid (e.g., CBD) and/or a modulator (e.g., memantine), optionally a supplement and optionally one or more excipients compressed into tablets or encapsulated in pharmaceutically acceptable capsules. In some embodiments, the mixtures or blends of dried particles or granules may include a coating of a cannabinoid and/or a modulator, a substrate, which may or may not comprise the same or a different active agent as the coating, and optionally one or more excipients, which may then be compressed into tablets or encapsulated in pharmaceutically acceptable capsules. In some embodiments, a pharmaceutical composition according to embodiments herein may be in the form of a nanoemulsion including at least one cannabinoid (e.g., CBD) and/or at least one modulator (e.g., memantine), optionally one or more excipient and optionally one or more supplement. An example nanoemulsion of a pharmaceutical composition according to embodiments herein may include a cannabinoid, a modulator, an excipient, a supplement, canola oil, soyabean oil, liquid paraffin, oleic acid, tween 20, tween 80, span 20, span 80, cetyl pyridinium chloride (CPC), glycerol, ethylene glycol, diethylene glycol monohexine ether, propylene glycol, bromophenol blue dye, tryptan blue, 3-(4,5-dimethylthiazol-2-yl)-2,5-diphenyl tetrazolium bromide (MTT), 1,1-diphenyl-2-picrylhydrazyl (DPPH), trypsin ethylenediaminetetraacetic acid (EDTA) and/or any combination of two or more of the foregoing.

### Methods of Preparation

Further described herein are methods of preparing pharmaceutical compositions for treating ophthalmic conditions according to various embodiments. Ophthalmic preparations suitable for contact on or near the surface of the eye, can be sterile, liquid, semi-solid, or solid and may contain one or more active pharmaceutical ingredient(s) (e.g., a cannabinoid, a modulator and/or a supplement) intended for application to the conjunctiva, the conjunctival sac, the eyelids and/or the areas around the eye. The choice of base and excipients used for the preparation of ophthalmic preparations may not affect adversely either the stability of the final product or the availability of the active ingredients at the site of action. In some embodiments, the ophthalmic preparations are free of coloring agents. Unless the active ingredient itself has antimicrobial activity, ophthalmic preparations supplied as multidose preparations may include a suitable antimicrobial agent. The antimicrobial activity may remain effective throughout the entire period of use. Ophthalmic preparations may include, but are not limited to, drops, emulsions, solutions, suspensions, ointments, lotions, gels and/or creams.

In one or more embodiments, eye drops are prepared to contain a pharmaceutical composition according to embodiments herein, a preservative (e.g., benzalkonium chloride, chlorbutanol, phenylmercuric acetate and/or phenylmercuric nitrate), a sterilizing agent (and/or subjected to a sterilization process such as autoclaving at about 100° C. to about 150° C., or about 121° C. for about 1 minute to about 60 minutes, or about 15 minutes or bacteria filtration), a tonicity agent (e.g., sodium chloride at about 0.1% to about 2%, or about 0.9%, boric acid at about 0.1% to about 5%, or about 1.9%), a buffer (e.g., boric acid, monobasic sodium phosphate) and/or a viscosity enhancer (e.g., a cellulose, methylcellulose, hydroxypropylmethylcellulose and/or polyvinyl alcohol). In some embodiments, a method of preparing eye drops may include forming a bactericidal and fungicidal vehicle by forming a solution of an aqueous-based (e.g., water, saline, normal saline) or oil-based (e.g., mineral oil, castor oil, flaxseed oil) solvent with one or more of phenyl mercuric nitrate/acetate at about 0.0005% to about 1%, or about 0.002%, a solution of benzalkonium chloride at about 0.001% to about 1%, or about 0.01% or a solution of chlorhexidine acetate at about 0.001% to about 1%, or about 0.01% dissolved therein. An active agent (e.g., in the form of a powder) such as a cannabinoid, a modulator and/or a supplement alone or in combination with excipients (e.g., a tonicity agent, a buffer and/or a viscosity enhancer) may be dissolved in the bactericidal and fungicidal vehicle to form a stable solution having the active ingredients at the desired concentrations. The stable solution may be clarified by passing the solution through a membrane filter (e.g., having a pore size of about 0.8 μm) and transferring and sealing the clarified solution in a storage container (e.g., a neutral glass container or a suitable plastic container). The eye drops may be sterilized by autoclaving or heating with a bactericide at about 40° C. to about 200° C., about 50° C. to about 100° C., or about 98° C. to about 100° C. for about 1 minute to about 60 minutes, or about 30 minutes or by filtration through a bacteria proof filter.

In one or more embodiments, an eye ointment is prepared to contain a pharmaceutical composition according to embodiments herein, a preservative (e.g., benzalkonium chloride, chlorbutanol, phenylmercuric acetate and/or phenylmercuric nitrate), a sterilizing agent (and/or subjected to a sterilization process such as autoclaving at about 100° C. to about 150° C., or about 121° C. for about 1 minute to about 60 minutes, or about 15 minutes or bacteria filtration), a tonicity agent (e.g., sodium chloride at about 0.1% to about 2%, or about 0.9%, boric acid at about 0.1% to about 5%, or about 1.9%), a buffer (e.g., boric acid, monobasic sodium phosphate) and/or a viscosity enhancer (e.g., a paraffin, a wool fat). In some embodiments, a method of preparing an eye ointment according to the disclosure includes melting wool fat (e.g., about 1 g to about 20 g, or about 10 g) and yellow soft paraffin (e.g., about 1 g to about 200 g, or about 80 g) on a water bath to form a melt solution. Adding liquid paraffin (e.g., about 1 g to about 20 g, or about 10 g) to the melt solution and mixing the components to form a base solution. The method may further include filtering the base solution through a coarse filter placed in a heated funnel. Sterilizing the filtered based solution using a dry heat method at about 100° C. to about 200° C., or about 160° C. for about 30 minutes to about 3 hours, or about 2 hours. Dissolving or dispersing the active agent (e.g., cannabinoid particles or solution, modulator particles or solution and/or supplement particles or solution) in the sterilized base solution to form the ointment.

Ophthalmic preparations containing dispersed solid particles may comply with the following test: a) take a quantity of the preparation (shake the container gently if necessary) corresponding to at least 10 μg of solid active ingredient (e.g., a cannabinoid, a modulator and/or a supplement) and place in a counting cell or spread in a thin layer on a slide; b) firmly apply a cover-glass and scan the whole area of the sample under a microscope; c) the whole sample may be scanned at low magnification (e.g. ×50) to identify particles >25 μm—larger particles may be measured at a higher magnification (e.g. ×200-×500); d) for each 10 μg of solid active substance, not more than 20 particles may have a maximum dimension greater than 25 μm and not more than two of these particles may have a maximum dimension greater than 50 μm—none of the particles should have a maximum dimension greater than 90 μm.

Nanoparticles, nanocrystals, nanocapsules and/or nanospheres (collectively referred to as nanounits) suitable for ophthalmic preparations and pharmaceutical compositions according to one or more embodiments herein, may be formed using solvent evaporation. Solvent evaporation may be used to prepare polymeric nanounits from a preformed polymer. Optionally, an oil-in-water (o/w) emulsion may be prepared, for example, in the preparation of nanospheres. The method can include preparing an organic phase comprising a polar organic solvent (e.g., ethyl acetate, dichloromethane, chloroform) and dissolving a polymer in the polar organic solvent to form a polymer solvent. Additionally, an active ingredient (e.g., a cannabinoid and/or a modulator) may be dissolved or dispersed within the polymer solvent to form the organic phase. In some embodiments, the method can include preparing an aqueous phase containing a surfactant (e.g., polyvinyl acetate, polyvinyl alcohol) and emulsifying the organic solution in the aqueous phase. The emulsified organic solution may then be processed using high-speed homogenization or ultrasonication, yielding a dispersion of nanodroplets. A suspension of nanounits may be formed by evaporating the polymer solvent, which may be allowed to diffuse through the continuous phase of the emulsion. The polymer solvent may be evaporated by, for example, continuous magnetic stirring at room temperature (in case of more polar solvents) or in a slow process of reduced pressure (as happens when using e.g., dichloromethane and chloroform). After the solvent has evaporated, the solidified nanounits can be washed and collected by centrifugation, followed by freeze-drying for long-term storage.

An emulsification/solvent diffusion method also may be used to prepare polymeric nanounits. The method can include the formation of an o/w emulsion between a partially water-miscible solvent containing a polymer and an active agent (e.g., a cannabinoid and/or a modulator), and an aqueous solution with a surfactant. The internal phase of this emulsion can include a partially hydro-miscible organic solvent, such as benzyl alcohol or ethyl acetate, which may be previously saturated with water in order to ensure an initial thermodynamic balance of both phases at room temperature. Subsequent dilution with a large amount of water can induce solvent diffusion from the dispersed droplets into the external phase, resulting in the formation of colloidal particles. Generally, this method may be used to produce nanospheres, but nanocapsules can also be obtained if a small amount of oil (such as triglycerides: C₆, C₈, C₁₀, C₁₂) is added to the organic phase. Depending on the boiling point of the organic solvent, this latter stage can be eliminated by evaporation or by filtration. The method can form nanounits with dimensions ranging from about 80 nm to 900 nm. This method can be applied to produce polymeric nanounits, despite the requirement of a high volume of the aqueous phase, which must be eliminated from the colloidal dispersion, and despite the risk of diffusion of a hydrophilic drug into the aqueous phase.

An emulsification/reverse salting-out method also may be used to prepare nanounits. The above described emulsification/solvent diffusion method can be considered a modification of the emulsification/reverse salting-out method. According to embodiments, the salting-out method is based on the separation of a hydro-miscible solvent from an aqueous solution, through a salting-out effect that can result in the formations of nanospheres. The main difference is the composition of the o/w emulsion, which can be formulated from a water-miscible polymer solvent, such as acetone or ethanol, and the aqueous phase contains a gel, the salting-out agent and a colloidal stabilizer. Examples of suitable salting-out agents include electrolytes, such as magnesium chloride (MgCl₂), calcium chloride (CaCl₂) or magnesium acetate [Mg(CH₃COO)₂], as well as non-electrolytes e.g., sucrose. The miscibility of acetone and water can be reduced by saturating the aqueous phase, which allows the formation of an o/w emulsion from the other miscible phases. The o/w emulsion is prepared, under intense stirring, at room temperature. Subsequently, the emulsion is diluted using an appropriate volume of deionized water or an aqueous solution in order to allow the diffusion of the organic solvent to the external phase, the precipitation of the polymer, and consequently, the formation of nanospheres. The remaining solvent and salting-out agent are eliminated by cross-flow filtration. The condition of complete miscibility between the organic solvent and water is not essential but simplifies the execution process. The dimensions of the nanounits obtained by this method can be about 170 nm to about 900 nm. The average size can be adjusted to values of about 200 nm and 500 nm, by varying polymer concentration of the internal phase/volume of the external phase.

In some embodiments, the method of preparation of a pharmaceutical composition according to embodiments herein may include a wet granulation process including premixing one or more active ingredient and excipients including a binder in a mixer to obtain a pre-mixture; granulating the pre-mixture of (1) by adding the granulation liquid, preferably purified water; drying the granules of (2) in a fluidized bed dryer or a drying oven; optionally dry sieving of the dried granules of (3); mixing the dried granules of (4) with the remaining excipients like glidant and lubricant in a mixer to obtain the final mixture; tableting the final mixture of (5) by compressing it on a suitable tablet press to produce tablets cores; (7) optionally film-coating of the tablet cores of (6) with a non-functional coat.

In some embodiments, the method of preparation of a pharmaceutical composition according to embodiments herein may include a direct compression process for making a pharmaceutical composition, wherein said process comprises the steps of: (1) premixing one or more active ingredient and the main portion of the excipients in a mixer to obtain a pre-mixture; (2) optionally dry screening the pre-mixture through a screen in order to segregate cohesive particles and to improve content uniformity; (3) mixing the pre-mixture of (1) or (2) in a mixer, optionally by adding remaining excipients to the mixture and continuing mixing; (4) tableting the final mixture of (3) by compressing it on a suitable tablet press to produce the tablet cores; (5) optionally film-coating of the tablet cores of (4) with a non-functional coat.

In some embodiments, the method of preparation of a pharmaceutical composition according to embodiments herein may include a dry granulation process for making a pharmaceutical composition, wherein said process comprises the steps of: (1) mixing one or more active ingredient with either all or a portion of the excipients in a mixer; (2) compacting the mixture of (1) on a suitable roller compactor; (3) reducing the ribbons obtained during (2) to granules, preferably small granules, by suitable milling or sieving steps; (4) optionally mixing the granules of (3) with the remaining excipients in a mixer to obtain the final mixture; (5) tabletting the granules of (3) or the final mixture of (4) by compressing it on a suitable tablet press to produce the tablet cores; (6) optionally film-coating of the tablet cores of (5) with a non-functional coat.

In one or more embodiments, further described herein are methods of preparing an ocular insert or punctal plug containing a pharmaceutical composition as described herein. The ocular insert or punctal plug may be sterile, thin, multilayered, drug-impregnated, solid and/or semisolid consistency devices that are placed into the cul-de-sac or conjunctival sac. A method of preparing an ocular insert such as a punctal plug may include providing a polymeric support that may or may not contain a pharmaceutical composition as described herein. The pharmaceutical composition can be incorporated subsequently as a dispersion or a solution on (i.e., as a film) or in (i.e., impregnated within) the polymeric support. The ocular insert or punctal plug may be soluble, insoluble or bio-erodible. Drug release from an insert or plug may depend on diffusion, osmosis and/or bio-erosion characteristics of the active ingredients in the pharmaceutical composition.

### Methods of Use

Further described herein are methods of using pharmaceutical compositions according to one or more embodiments herein. In some embodiments, the methods include treating an ophthalmic condition with one or more embodiments of a pharmaceutical composition as described herein. Ophthalmic conditions treatable by pharmaceutical compositions according to embodiments herein may include, but are not limited to, those involving inflammation, pain, fibrosis, infection, virus, parasite, neurodegeneration, retinal ganglion cell (RGC) degeneration and/or combinations thereof. In some embodiments, the ophthalmic condition affects the eyelid, ocular surface, back of the eye, dermis, eyelashes, tarsal conjunctiva, mucocutaneous junction and/or meibomian glands. In some embodiments, the ophthalmic condition partially caused by bacteria, virus, fungus, yeast, mites, seborrhea, rosacea, environmental exposure, hormonal dysregulation, inflammatory process, cytokine cascade and/or an autoimmune disorder.

Suitable ophthalmic conditions that may be treated with pharmaceutical compositions according to one or more embodiments herein may include, but are not limited to, blepharitis, glaucoma, uveitis, a stye, rosacea, autoimmune disease, glandular condition, chalazion, corneal abrasion, corneal herpetic infections, corneal ulcer, dry eyes, iritis, keratitis, optic neuritis, inflammation secondary to an external environmental exposure, conjunctivitis, scleritis, age-related macular degeneration, allergic conjunctivitis, eosinophilic keratitis, anterior segment scarring, blepharoconjunctivitis, a bullous disorder, cicatricial pemphigoid, conjunctival melanoma, contact lens-associated giant papillary conjunctivitis, diabetic retinopathy, episcleritis, gliosis, granuloma annulare, Graves' ophthalmopathy, intraocular melanoma, keratitis, keratoconjunctivitis pain, pinguecula, post-surgical pain, inflammation, proliferative vitreoretinopathy, pterygia, Sjögren's syndrome, Scleroderma, systemic sclerosis, vernal and/or any combination of two or more of the foregoing. In certain embodiments, the method includes treating an ophthalmic inflammatory disorder, for example, blepharitis, conjunctivitis, iritis, uveitis, keratitis or corneal ulcer. In certain embodiments, the ophthalmic condition treatable by a pharmaceutical composition according to embodiments herein is caused by a bacteria, for example, Staphylococcus aureus. In certain embodiments, the ophthalmic condition treatable by a pharmaceutical composition according to embodiments herein is caused by an autoimmune disorder, for example, rosacea. In certain embodiments, the ophthalmic condition treatable by a pharmaceutical composition according to embodiments herein is caused by environmental exposure, for example, from sarin gas, agent orange, botulinum toxin or novichuk agent. In some embodiments the methods include further treating secondary wound and/or pain.

In some embodiments, methods of treatment with a pharmaceutical composition according to embodiments herein, including treating one or more of watery eye, red eye, gritty sensation in eye, burning sensation in eye, stinging sensation in eye, itchy eyelid, red eyelid, swollen eyelid, flaking of skin around the eye, crusted eyelash, eyelid sticking, frequent blinking, sensitivity to light and/or blurred vision.

In some embodiments, the methods of treatment include administering to a subject at least one cannabinoid according to one or more embodiments described herein. The method may alternatively or further include administering at least one modulator compound according to one or more embodiments described herein, for example, where the modulator acts as one or more of an NMDA receptor modulator, a 5-HT₃ receptor modulator, a nicotinic acetylcholine receptor modulator, a dopamine D2 receptor modulator, or a sigma-1 receptor modulator.

According to one or more embodiments, the methods of treatment can include administering to a subject a pharmaceutical composition according to embodiments described herein to treat or prevent blepharitis. Blepharitic changes limited primarily to the posterior lid margin arise predominantly from pathological processes centered around the meibomian glands. “Secondary meibomitis” represents a localized inflammatory response in which the meibomian glands are secondarily inflamed in a spotty fashion from an anterior lid margin blepharitis. Both “meibomian seborrhea” and “primary meibomitis” produce generalized gland dysfunction. Acne rosacea, seborrheic dermatitis, psoriasis, atopy and hypersensitivity to bacterial products may all contribute to the etiology of blepharitis. It is generally assumed that infection plays a role in anterior blepharitis. Chronic blepharitis may have an inflammatory etiology that is not associated with infection.

In some embodiments, a pharmaceutical composition administered for treating blepharitis includes administering a cannabinoid (e.g., CBD) and/or a modulator (e.g., memantine) to a subject in an effective amount to treat the blepharitis and associated symptoms including inflammation and pain. In certain embodiments, the pharmaceutical composition may be in the form of eye drops or a topical ointment, for example, containing nanounits or a nanoemulsion of the cannabinoid and/or modulator.

### Kits

Further described herein are kits containing a pharmaceutical composition according to one or more embodiments. In some embodiments, a kit may include a cannabinoid dosage form as described herein, a modulator dosage form as described herein and optionally a supplement dosage form as described herein. The kit may further include instructions for administering the dosage forms in a suitable manner to achieve a desired therapeutic effect for treating an ophthalmic condition. For example, the instructions may direct a subject to administer the dosage forms concurrently, simultaneously or sequentially according to a dosing schedule. The dosage forms may be independently in the form of ophthalmic preparations (e.g., eye drops, eye ointment, etc.), solid oral dosage forms, inserts, plugs, lenses or in any suitable form as described herein.

In some embodiments, a kit may include tablets or capsules, each tablet or capsule comprising a cannabinoid, a modulator and optionally a supplement. The kit may further include instructions for administering the tablet or capsule in a suitable manner to achieve a desired therapeutic effect for treating an ophthalmic condition. For example, the instructions may direct a subject to administer the tablet or capsule according to a dosing schedule.

### EXAMPLES

### Example 1 (Prophetic)—Eye Drops Containing CBD and Memantine

Eye drops are prepared by forming a bactericidal and fungicidal vehicle containing a solution of normal saline and benzalkonium chloride at about 0.001% to about 1%, or about 0.1%. A powder formed of CBD nanoparticles and a powder formed of memantine nanoparticles are dispersed in the bactericidal and fungicidal vehicle. Sodium chloride at about 0.1% to about 2% and/or about 0.9%, boric acid at about 0.1% to about 5%, or about 1.9%, boric acid and/or monobasic sodium phosphate and methylcellulose, hydroxypropylmethylcellulose and/or polyvinyl alcohol are dissolved in the bactericidal and fungicidal vehicle to form a stable solution having the CBD and memantine each individually at a concentration of about 0.1 wt % to about 20 wt %. The stable solution is clarified by passing the solution through a membrane filter having a pore size of about 0.8 μm. The clarified solution is sterilized in an autoclave at about 98° C. to about 100° C. for about 30 minutes. The resulting eye drops may have the composition as shown in Table 1.

TABLE 1
Prophetic Eye Drop Formulation

| | Concentration |
|---|---|
| | per unit dose |
| Component | (wt %) |
| Cannabidiol | 0.05-2% |
| Memantine | 0.05-2% |
| Benzalkonium Chloride | 0.1% |
| Sodium Chloride | 0.9% |
| Boric Acid | 1.9% |
| Hydroxypropyl Methylcellulose | 2.0% |
| Water | QS to 10 mL |

An eye ointment is prepared by melting about 10 g wool fat and about 80 g of yellow soft paraffin on a water bath. Adding about 6 g of liquid paraffin to the melt solution and mixing the components to form a base solution. The base solution is filtered through a coarse filter seated in a heated funnel. The filtered base solution is sterilized using a dry heat method at about 160° C. for about 2 hours. Cannabinoid nanoparticles and memantine nanounits are dispersed in the sterilized base solution to form the ointment. The resulting ointment may have the composition as shown in Table 2.

TABLE 2
Prophetic Ointment Formulation

| | Concentration |
|---|---|
| | per unit dose |
| Component | (wt %) |
| Cannabidiol | 2% |
| Memantine | 2% |
| Wool fat | 10% |
| Yellow soft paraffin | 80% |
| Liquid paraffin | 6% |

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A method of treating an ophthalmic condition comprising the step of administering to a subject i) a cannabinoid, and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator.

64. The method of claim 63, wherein the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator.

65. The method of claim 63, wherein the modulator compound is an NMDA receptor antagonist.

66. The method of claim 63, wherein the modulator compound is glutamatergic NMDA receptor antagonist.

67. The method of claim 63, wherein the modulator compound is 5-HT₃ receptor antagonist.

68. The method of claim 63, wherein the modulator compound is a nicotinic acetylcholine receptor antagonist.

69. The method of claim 63, wherein the modulator compound is a dopamine D2 receptor agonist.

70. A pharmaceutical composition comprising i) a cannabinoid and ii) a modulator compound that acts as one or more of an NMDA receptor modulator, a 5-HT₃ receptor modulator, a nicotinic acetylcholine receptor modulator, a dopamine D2 receptor modulator, or a sigma-1 receptor modulator.

71. The pharmaceutical composition of claim 70, wherein the NMDA receptor modulator is an agonist, an antagonist, or a mixed agonist-antagonist at one or more of the NMDA receptor modulator, the 5-HT₃ receptor modulator, the nicotinic acetylcholine receptor modulator, the dopamine D2 receptor modulator, or the sigma-1 receptor modulator.

72. The pharmaceutical composition of claim 70, wherein the modulator compound is an NMDA receptor antagonist.

73. The pharmaceutical composition of claim 70, wherein the modulator compound is glutamatergic NMDA receptor antagonist.

74. The pharmaceutical composition of claim 70, wherein the modulator compound is 5-HT₃ receptor antagonist.

75. The pharmaceutical composition of claim 70, wherein the modulator compound is a nicotinic acetylcholine receptor antagonist.

76. The pharmaceutical composition of claim 70, wherein the modulator compound is a dopamine D2 receptor agonist.

77. The pharmaceutical composition of claim 70, wherein the modulator compound is a sigma-1 receptor agonist.

78. The pharmaceutical composition of claim 70, wherein the modulator compound is memantine, a prodrug thereof, an analog thereof, a metabolite thereof or a pharmaceutically acceptable salt thereof.

79. The pharmaceutical composition of any of claim 70, wherein the cannabinoid comprises one or more of a natural compound, a synthetic compound, or a semi-synthetic compound.

80. The pharmaceutical composition of any of claim 70, wherein the cannabinoid is at least one of an anti-inflammatory, anti-fibrotic, anti-infective or neuroprotective agent.

81. The pharmaceutical composition of any of claim 70, wherein the cannabinoid comprises one or more of cannabinol, cannabidiol, cannabigerol, cannabichromene, Δ9-tetrahydrocannabinol, Δ8-tetrahydrocannabinol, 11-hydroxy-tetrahydrocannabinol, 11-hydroxy-Δ9-tetrahydrocannabinol, levonantradol, Δ11-tetrahydrocannabinol, tetrahydrocannabivarin, dronabinol, amandamide, nabilone, a varin thereof, prodrug thereof, an analog thereof, a metabolite thereof or a pharmaceutically acceptable salt thereof.

82. The pharmaceutical composition of any of claim 70, wherein the cannabinoid is cannabidiol hemisuccinate.
